(19) United States
(12) Patent Application Publication
(10) Pub. No.: US 2011/0168176 A1
(43) Pub. Date: Jul. 14, 2011

(54) RESPIRATOR OR ANESTHESIA APPARATUS

(75) Inventors: Grigory KHOLTCHANSKI, Lubeck (DE); Robert LISCHINSKI, Einhaus (DE); Sven PASDZIOR, Lubeck--Travemunde (DE); Klaus RADOMSKI, Lubeck (DE); Dirk-Stefan REICHERT, Lubeck (DE)

(73) Assignee: DRAGER MEDICAL GMBH, Lubeck (DE)

(21) Appl. No.: 12/981,804

(22) Filed: Dec. 30, 2010

(30) Foreign Application Priority Data
Jan. 14, 2010 (DE) 10 2010 004 527.6

(51) Int. Cl. A61M 16/01

(52) U.S. Cl. 128/203.12

(57) ABSTRACT

A pneumatic connection for connecting two modules (6) with one another into a simple and reliable pneumatic connection of modules (6) of the respirator or anesthesia apparatus (5). A small installation space needed is provided in a respirator or anesthesia apparatus (5), including at least two modules (6), e.g., a blower, a breathing system, a mixer and/or an anesthetic evaporator (11, 12, 13, 14). The at least one pneumatic connection includes a connection tube (1), which is surrounded with a first end (9) by a first module (7) and with a second end (10) by a second module (8), so that there is a pneumatic connection through the connection tube (1) between the first module (7) and second module (8). The reliable pneumatic connection is also established when the modules (6) have an offset in their alignment in relation to one another.

### CROSS REFERENCE TO RELATED APPLICATIONS

This application claims the benefit of priority under 35 U.S.C. §119 of German Patent Application DE 10 2010 004 527.6 filed Jan. 14, 2010, the entire contents of which are incorporated herein by reference.

### FIELD OF THE INVENTION

The present invention pertains to a respirator or anesthesia device comprising at least two modules, e.g., a blower, a breathing system, a mixer and/or an anesthetic evaporator and at least one pneumatic connection for the pneumatic connection of two modules.

### BACKGROUND OF THE INVENTION

Respirators and anesthesia apparatuses are used in medical technology for the artificial respiration of patients and administering anesthesia to patients. The respirators and anesthesia apparatuses comprise here a plurality of modules, which are connected to one another in a pneumatically gastight manner by means of a pneumatic connection means. The modules are, for example, a blower, a mixer, an anesthetic evaporator or a breathing system.

A breathing tube for respirating a patient is connected to the breathing system. During manufacture, the individual modules of the respirator or anesthesia apparatus are manufactured at first separately and independently from one another and are subsequently to be connected to one another by the pneumatic connection means. In general, two modules are connected by a plurality of pneumatic connection means, because a plurality of connections are, as a rule, necessary between two modules for the operation of the respirator or anesthesia apparatus.

Flexible tubes or, in some applications, even tube connections are used, in general, as pneumatic connection means.

Such tube connections are connected sealingly to the modules usually by means of O-rings.

The large number of necessary connections in a respirator or anesthesia apparatus and hence of flexible tubes and/or tube connections leads to a great assembly effort for connecting the modules to one another.

The tube connections must be mounted in an exact orientation to the modules in order for a tight connection to be guaranteed indirectly via the O-rings. Only small mounting tolerances of the modules in relation to one another and of the modules in relation to the corresponding tube connections are permissible in the vertical, horizontal and axial positions.

After the modules have been connected to the flexible tubes and/or tube connections, a complicated test is to be carried out in order to identify leaks or flexible tube connections plugged incorrectly into one another, because this represents a considerable potential hazard to the patients.

The flexible tubes with corresponding connection pieces at the ends must have a certain minimum length as a function of the diameter, as well as as a function of the radii of curvature in order for the ends of the flexible tubes to be able to be plugged into the ports of the modules.

In case of short distances between two modules, this is only possible, in general with a large loop, which causes a great length of the flexible tube with a correspondingly high pneumatic resistance and dead space to be necessary. In addition, a relatively great minimum distance is necessary between the modules to be connected.

U.S. Pat. No. 3,394,954 shows a coupling device for medical devices for connecting two flexible tubes. A flexible tube is disadvantageously necessary herewith.

U.S. Pat. No. 4,870,961 shows a pneumatic connection means for connecting a respiration tube with a medical ventilator.

### SUMMARY OF THE INVENTION

The object of the present invention is therefore to make available a respirator or anesthesia apparatus, in which the modules of the respirator or anesthesia apparatus can be pneumatically connected to one another in a simple and reliable manner and the space requirement is small.

This object is accomplished with a respirator or anesthesia apparatus comprising at least two modules, e.g., a blower, a breathing system, a mixer and/or an anesthetic evaporator, at least one pneumatic connection means for the pneumatic connection of two modules, wherein the at least one pneumatic connection means comprises a connection tube, which is surrounded with a first end by a first module and is surrounded with a second end by a second module, so that there is a pneumatic connection through the connection tube between the first module and the second module.

The modules of a respirator or anesthesia apparatus can thus be connected to one another by the connection tube in a simple manner without a flexible tube.

Secure and reliable pneumatic connection is thus possible between two modules, and reliable pneumatic connection can be achieved with a small amount of assembly effort.

Furthermore, the necessary distance between the two modules for the pneumatic connection is small, so that the amount of space needed for the installation of the respirator or anesthesia apparatus can be reduced as a result.

Furthermore, unlike in a flexible tube connection, no flexible tubes are necessary, which leads to a reduction of pneumatic resistances and dead spaces in the gas-carrying system of the respirator or anesthesia apparatus. In addition, mix-up of flexible tube plug connections among each other is avoided.

The connection tube is, in particular, a separate component and/or not a flexible tube. The connection tube is a separate component, i.e., the connection tube is not connected in substance with at least one module. The connection tube thus represents a separate component, which is manufactured separately and independently from the module.

In another embodiment, the connection tube is connected to the first module and/or to the second module in a nonpositive and/or positive-locking manner.

In an additional embodiment, the first and/or second module has a recess, and the first end of the connection tube is arranged in the recess of the first module and/or the second end of the connection tube is arranged in the recess of the second module. The recess is designed here, for example, as a hole or milled recess in a connection tube, which has a round cross section.

The connection tube preferably consists of an inner material on the inside and an outer material on the outside. The inner tube thus has an inner tube consisting of an inner material and an outer tube consisting of an outer material. The outer tube is preferably fastened to the inner tube by connection in substance, for example, by means of vulcanization.

In addition, the outer tube may also be fastened to the inner tube, for example, by means of bonding.

In one variant, the inner material has a higher modulus of elasticity than the outer material.

The modulus of elasticity of the inner material is preferably 2, 5, 10, 30 or 50 times higher than the modulus of elasticity of the outer material.

The outer material is preferably formed from an elastic material, for example, an elastomer, which is resistant to corrosive media such as anesthetic gases (laughing gas, O₂) and volatile anesthetics such as halothane, enflurane, desflurane, isoflurane or sevoflurane. Suitable materials resistant to the aforementioned media are, for example, materials from the group of the ethylene, propylene and rubber compounds and of the ethylene and propylene elastomers (EPDM), which are known, for example, under the trademarks Vistalon®, Dutral®, Buna AP® or APTK®, as well as of the fluorinated rubber compounds (FKM, FPM), which are known under the trademarks Viton®, Tecnoflon®, and Fluorel®, as well as of the perfluorinated rubber compounds (FFKM, FFPM), which are known under the trademarks Parafluor® and Kalrez®.

In another embodiment, the inner material is a corrosion-resistant metal, especially stainless steel. The gas of the respirator or anesthesia apparatus flows through the inner tube. It is meaningful to make the inner tube from stainless steel because a corrosion-resistant and durable flow duct resistant to corrosive media, especially to volatile anesthetics such as halothane, isoflurane, desflurane, sevoflurane or enflurane can be made available as a result for transporting the gas.

Making the inner material from polyether ether ketone (PEEK) guarantees resistance to corrosive and aggressive media, which are represented, for example, by volatile anesthetics such as halothane, isoflurane, desflurane, sevoflurane or enflurane, in an equivalent and similar form as it is guaranteed by making the inner material from stainless steel.

In another embodiment, an especially elastic sealing bead is formed at the first end and/or at the second end of the connection tube, preferably with the outer material. The sealing bead is preferably designed as a fully circumferential sealing bead.

It is also preferable to make the sealing bead from the same elastic material as the outer material, and more preferably from ethylene or propylene rubber (EPDM), fluorinated rubber (FKM, FPM) or perfluorinated rubber (FFKM, FFPM).

In another embodiment, the connection tube makes possible a radial and/or axial sealing of the connection tube in relation to the module, especially of the first and/or second end in a recess of the module, at the first end and/or at the second end, especially by means of the sealing bead.

In another embodiment, the sealing bead is in contact with the first and/or second module, especially in a recess, so that a sealing surface is formed between the sealing bead and the first and/or second module.

In an additional variant, the connection tube has a round cross section.

In another variant, the cross-sectional shape of the recess of the first and/or second module corresponds to the cross-sectional shape of the connection tube at the first end, especially at the sealing bead.

The outer shape of the connection tube at the first and/or second end and the shape of the recess are of an essentially identical design, so that a circumferential and complete sealing surface can form.

A round design of the connection tube and of the recesses in the modules guarantees, in particular, that both a horizontal, a vertical, as well as an angular offset between the modules can be compensated.

In another embodiment, the cross-sectional shape of the recess of the first and/or second module corresponds to the outer cross-sectional shape of the connection tube at the first and/or second end, especially the sealing bead.

The first and/or second end, especially the sealing bead of the connection tube, is slightly larger than the recess of the second module in a state in which it is not inserted into the recess of the first module, so that the first and/or second end of the connection tube is arranged in the recess of the first and/or second module under a prestress.

The first and/or second end of the connection tube, especially the elastic sealing bead, are thus arranged in the recess of the first and/or second module under an elastic prestress. The prestress is brought about by an elastic deformation of the elastic sealing bead. As a result, a compressive strain develops at the sealing surface between the first and/or second end of the connection tube and the limitation of the recess by the first and/or second module, so that secure and reliable sealing is guaranteed as a result between the first and/or second end of the connection tube and the first and/or second module at the recess. Furthermore, manufacturing inaccuracies at the ends of the connection tubes and at the recesses of the modules can also be advantageously compensated based on this elastic deformability.

In another embodiment, the sealing bead is embodied coaxially with an axis of the first and/or second connection tube.

In another embodiment, the first and/or second connection tube is arranged coaxially in the recess of the first and/or second module.

Exemplary embodiments of the present invention will be described in more detail below with reference to the drawings attached. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a greatly simplified longitudinal sectional view of a respirator with two modules;

FIG. 2 is a greatly simplified longitudinal sectional view of an anesthesia apparatus with four modules;

FIG. 3 is a longitudinal sectional view of a connection tube in a first exemplary embodiment in a recess of a second module;

FIG. 4 is a longitudinal sectional view of the connection tube in a second exemplary embodiment in the recess of the second module;

FIG. 5 is a longitudinal sectional view of the connection tube in a third exemplary embodiment in the recess of the second module;

FIG. 6a is a longitudinal sectional view of a connection tube and of two modules in a first mounting possibility in detail with dimensions;

FIG. 6b is a longitudinal sectional view of two connection tubes and of a first and second module in a first mounting possibility;

FIG. 7 is a longitudinal sectional view of two connection tubes and of the first and second module in a second mounting possibility;

FIG. 8 is a longitudinal sectional view of two connection tubes and of the first and second module in a third mounting possibility;

FIG. 9 is a longitudinal sectional view of two connection tubes and of the first and second modules in a fourth mounting possibility; and

FIG. 10 is a longitudinal sectional view of a connection tube and of the first and second module in a fifth mounting possibility.

### DESCRIPTION OF THE PREFERRED EMBODIMENTS

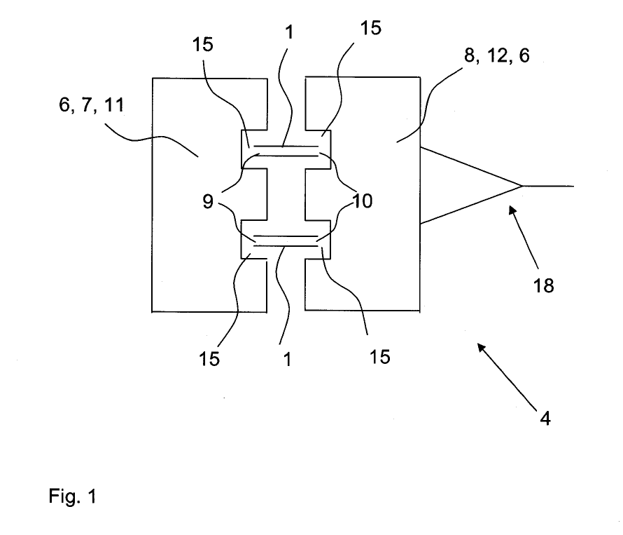

Referring to the drawings in particular, FIG. 1 shows a respirator 4 in a highly simplified manner. Respirator 4 comprises two modules 6, namely, a first module 7 and a second module 8. The two modules 6 are connected to one another pneumatically by two connection tubes 1. Each module 6 has two recesses 15 each, in which an end 9, 10 each of the connection tube 1 is arranged. The first module 7 is a blower 11 and the second module 8 is a breathing system 12. A flexible breathing tube 18 is connected to the breathing system 12.

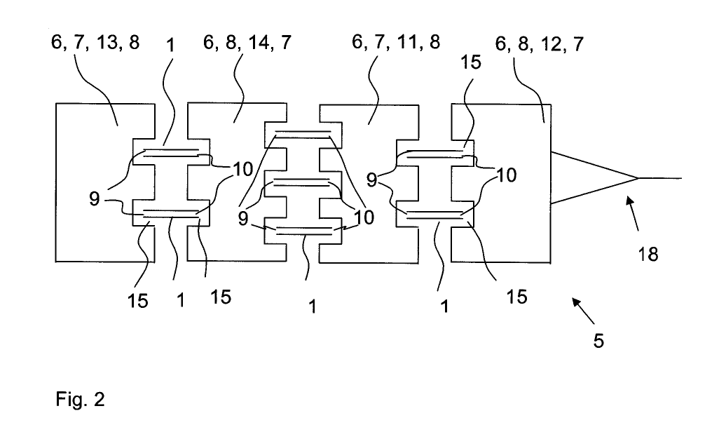

Analogously to FIG. 1, FIG. 2 shows an anesthesia apparatus 5. The anesthesia apparatus 5 comprises four modules 6, which are pneumatically connected to one another by means of connection tubes 1 analogously to the first exemplary embodiment according to FIG. 1. The connection tubes 1 are arranged at their respective ends 9, 10 in recesses 15 of the four modules 6 of the anesthesia apparatus 5. The anesthesia apparatus 5 comprises, besides the blower 11 and the breathing system 12, a mixer 13 and an anesthetic evaporator 14. The anesthetic evaporator 14 can be connected to an anesthesia or anesthetic dispensing unit. The flexible breathing tube 18 for the artificial respiration of the patient is connected to the breathing system 12.

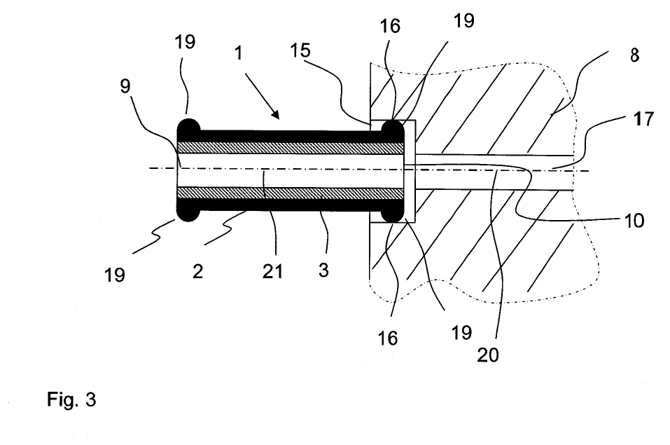

FIG. 3 through FIG. 5 show three different exemplary embodiments for the connection tube 1. Only the second module 8 is shown in FIGS. 3 through 5. The first module 7 is not shown in FIGS. 3 through 5. In addition, a symmetry line is shown axially symmetrically to the air line 17 in the second module 8 and a symmetry line 21 is shown axially symmetrically to the connection tube 1. The symmetry lines 20, 21 are obtained as a single symmetry line that is identical in space in the straight alignment shown of the air line 17 of the second module 8 with the connection tube 1.

In the first exemplary embodiment of the connection tube 1 according to FIG. 3, the connection tube 1 has an outer tube 2 consisting of an elastic outer material and an inner tube 3 consisting of a non-elastic inner material. The outer tube 2 is attached to the inner tube 3 in substance by means of vulcanization. The connection tube 1 has a first end 9 and a second end 10. The second end 10 of the connection tube 1 is arranged in the recess 15 of the second module 8. A round sealing bead 19 consisting of the outer material, namely, the elastomer, is formed at the second and first ends 9, 10 of the connection tube 1. The connection tube 1 has a round cross section and recess 15 is a hole having a round cross section. The outer cross-sectional shape of the second end 10 of the connection tube 1, namely, especially the sealing bead 19 at the second end 10, thus corresponds to the cross-sectional shape of the recess 15 having a round cross section. As a result, when the connection tube 1 is inserted into the recess 15, a fully circumferential sealing surface 16 can be prepared between the sealing bead 19 and the second module 8 at the recess 15.

In a state of the connection tube 1 in which it is not inserted into the recess 15, the sealing bead 19 is slightly larger than recess 15, so that the sealing bead 19 is elastically deformed when it is inserted into the recess 15 on account of its elastic properties, i.e., the circumference of the sealing bead 19 is reduced. The sealing bead 19 is thus arranged in the recess 15 under a prestress, so that secure and reliable sealing is guaranteed between the sealing bead 19 and the second module 8 at the recess 15 based on the acting compressive forces. Furthermore, manufacturing inaccuracies on the connection tube 1 and on the recess 15 can be compensated as a result. The first end 9 of the connection tube 1 is arranged analogously to the second end 10 in a recess 15 of the first module 7 (not shown). The air can thus flow, for example, from the second module 8 through an air line 1, i.e., through the space connected by the inner tube 3, and subsequently into an air line 17 of the first module 7 according to FIG. 3, not shown. The sealing bead 19 according to the first exemplary embodiment in FIG. 3 makes possible a radial sealing of the connection tube 1 against the second module 8 at recess 15 based on the geometry and the arrangement in recess 15.

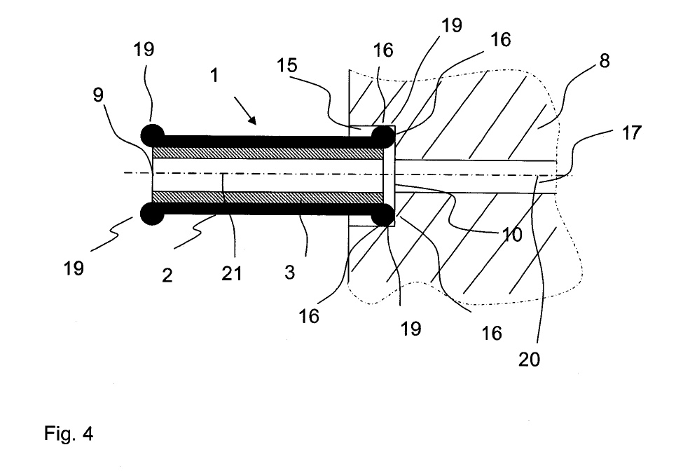

The second exemplary embodiment according to FIG. 4 differs from the first exemplary embodiment of the connection tube 1 only by the geometry of the sealing bead 19. The sealing bead 19 is extended outwardly in the axial direction relative to the inner tube 3, so that an axial sealing surface 16 can also be prepared, besides a radial sealing surface 16, by means of the sealing bead 19. The second exemplary embodiment otherwise corresponds to the first exemplary embodiment according to FIG. 3. Identical components in the second exemplary embodiment according to FIG. 4 are the same as in the first exemplary embodiment according to FIG. 1 and are designated by the same reference numbers as in FIG. 1.

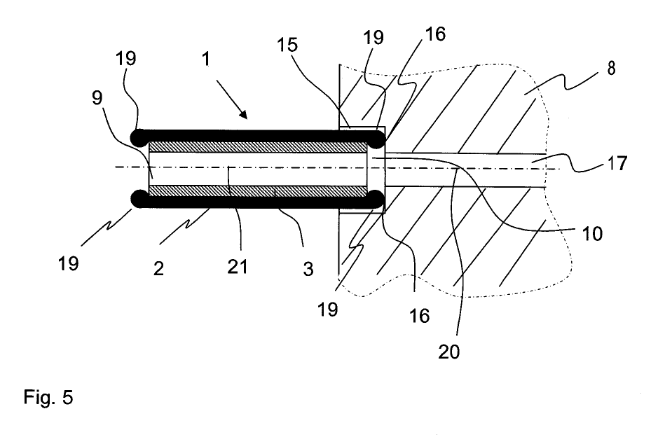

The third exemplary embodiment according to FIG. 5 differs from the first exemplary embodiment according to FIG. 3, in turn, only in the geometry of the sealing bead 19. The sealing bead 19 is axially extended and radially offset inwardly relative to the inner tube 3, so that only an axial sealing surface 16 can be prepared as a result by means of the sealing bead 19 in recess 15. The third exemplary embodiment according to FIG. 5 otherwise corresponds to the first exemplary embodiment of the connection tube 1 according to FIG. 3. Identical components in the third exemplary embodiment according to FIG. 5 are the same as in the first exemplary embodiment according to FIG. 1 and are designated by the same reference numbers as in FIG. 1.

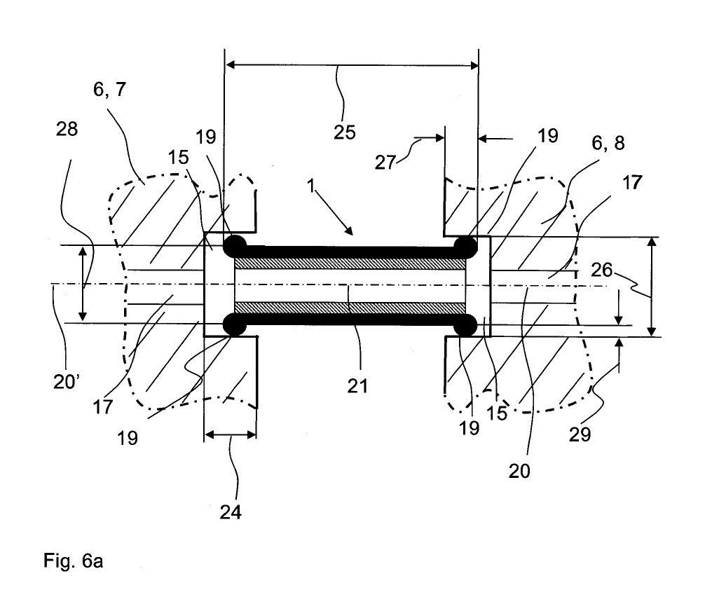

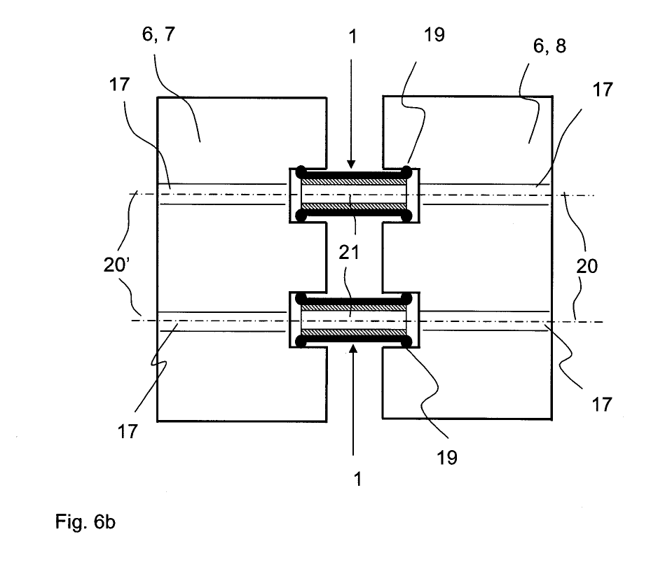

FIGS. 6a through 10 show five different mounting situations of a first and second module 7, 8 with two connection tubes 1 each. The connection tubes 1 with sealing beads 19 are arranged in recesses 15 of the modules 7, 8 and connect the air lines 17 of the modules 6, 7, 8 with one another. Horizontal symmetry lines 20, 20′, 21 are shown axially symmetrically with the respective air lines 17 in modules 6, 7, 8 and with the connection tubes 1 in FIGS. 6 through 10 in order to more specifically illustrate in detail the positions of the modules 6, 7, 8 in relation to one another as well as the positions of modules 6, 7, 8 in relation to the connection tubes 1.

For representation in detail, only a single connection tube 1 is shown in the arrangement according to FIG. 6a in an arrangement comprising the first module 7 and the second module 8 in a straight connection without height offset or angular offset in the arrangement of the modules 7, 8 relative to one another. The sealing beads 19 seal in the recesses 15 such that a gastight connection is obtained between the air lines 17 of the first and second modules 7, 8. The conditions in space of a connection tube 1, modules 6, sealing bead 19, air line 17 and recesses 15 are shown as an example in FIG. 6a in a view that is not true to scale.

The connection tube 1 is in a recess with a height 26, and it is fitted in the vertical position into recess 15 of module 6 with its maximum external diameter, comprising a tube diameter 28 of the connection tube 1 and the sealing bead thickness 29 of the sealing bead 19. The connection tube 1 is fitted over the tube length 25 into the recess depth 24 with a plug-in depth 27 in the horizontal position in the recess 15 of connection tube 1.

As an example of a connection comprising the modules 6 by means of a connection tube 1, a tube length 25, equaling 35 mm, a recess depth 24, equaling 11 mm and a plug-in depth 27, equaling 5 mm, shall be dimensioned such that the connection tube 1 is fitted horizontally centrally into the recess 15. A recess height 26, equaling 14 mm, shall be dimensioned such that the connection tube 1 with a tube diameter 28 of 10 mm and with a sealing bead thickness 29 of 2.1 mm to 2.4 mm is sealingly fitted into the recess 15.

In the view in FIG. 6b, there is no tolerance, i.e., no height offset or angular offset in the geometric arrangement in relation to one another between the first module 7 and the second module 8. A connection with two connection tubes 1 is shown. Secure and reliable pneumatic connection is given between the first module 7 and the second module 8.

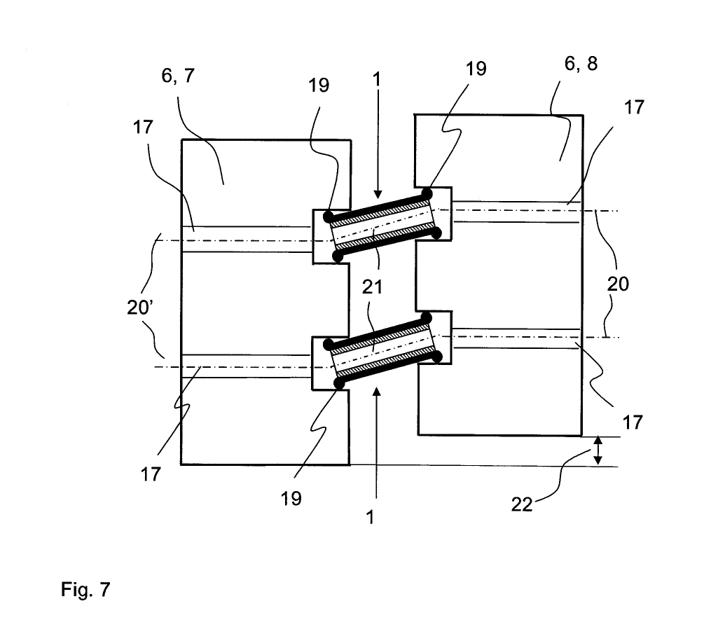

In the mounting possibility according to FIG. 7, the second module 8 is arranged slightly higher than the first module 7, i.e., there consequently is a height offset 22 between the second module 8 and the first module 7. A connection with two connection tubes 1 is shown. Based on the elastic design of the sealing bead 19, this mounting possibility also makes possible a secure and reliable pneumatic connection between the first module 7 and the second module 8.

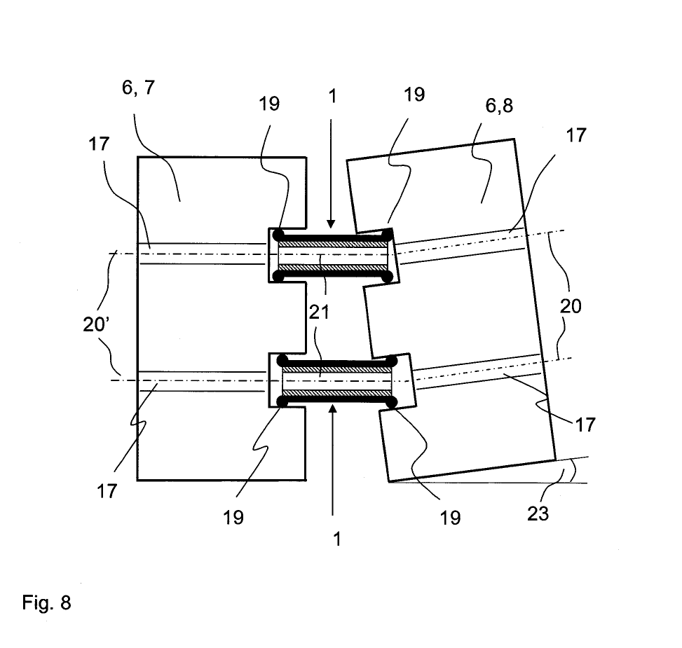

There is an angular offset 23 between the first module 7 and the second module 8 in FIG. 8. This means that the distance between the first and second modules 7, 8 is smaller at the top than that between the first and second modules 7, 8 at the bottom. A connection with two connection tubes 1 is shown. Reliable pneumatic connection is guaranteed between the first and second modules 7, 8 in this installation situation or mounting possibility as well.

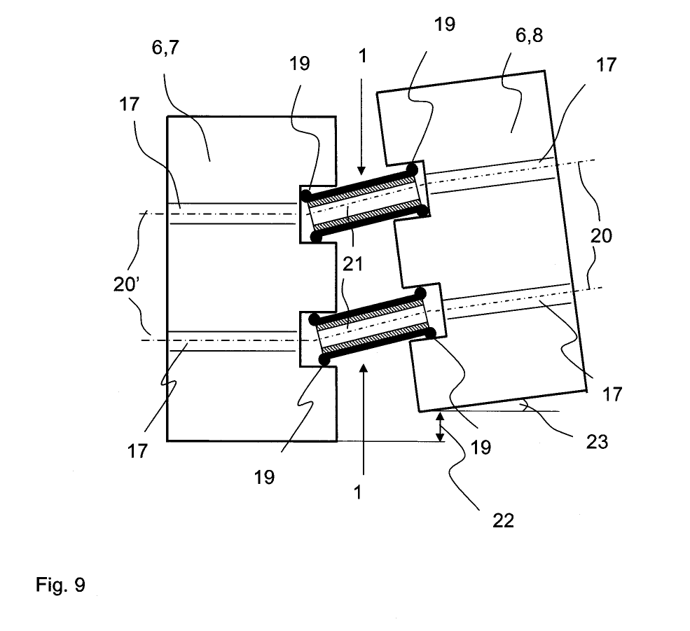

In the fourth mounting possibility according to FIG. 9, there is both a height offset 22 and an angular offset 23 between the first and second modules 7, 8. A connection with two connection tubes 1 is shown. The distance between the first and second modules 7, 8 is smaller here at the top than at the bottom and, furthermore, the second module 8 is arranged slightly higher than the first module 7. There is a reliable pneumatic connection between the first and second modules 7, 8 in this mounting possibility as well.

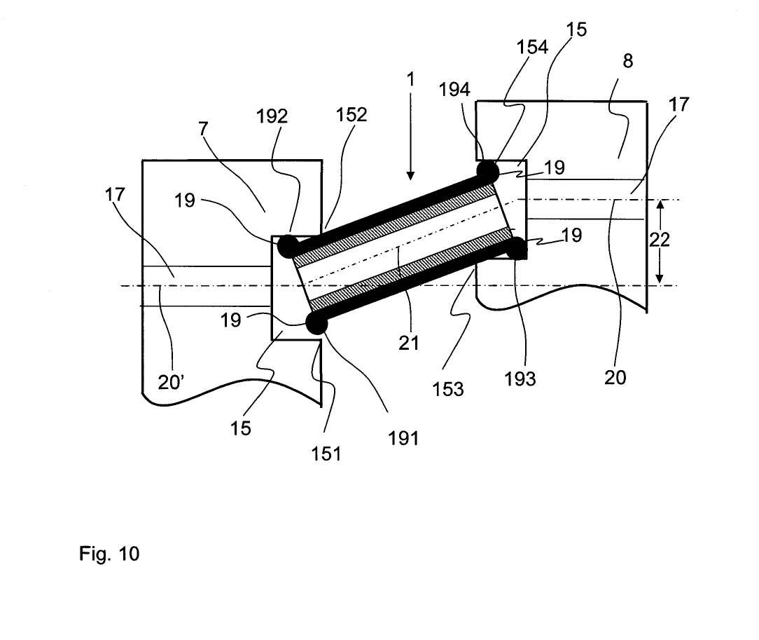

To illustrate the structural limitations of the connection of a first module 7 with a second module 8, the fifth mounting possibility according to FIG. 10 shows an embodiment with an excessive height offset 22. The distance between the first and second modules 7, 8 is smaller at the top than at the bottom. The second module 8 is arranged in this case more than slightly higher than the first module 7.

To highlight the details of the arrangement, a connection with only one connection tube 1 is shown, in which the arrangement exceeds the structurally imposed limitations, i.e., the height offset 22 cannot be compensated any longer. Contact points 151, 152, 153, 154 of the recesses 15 and contact points 191, 192, 193, 194 of the sealing beads 19 are shown, whose positions in relation to one another show whether a sealing connection is possible. It can be seen that at least at contact point 153 of the recess 15 of the second module 8, the connection tube 1 is shown in the tilted position and with overlap in relation to the second module, and that the connection tube 1 cannot be fitted any longer into recess 15 without a mechanical deformation of the connection tube 1 and/or of the second module 8. The consequence of this is that the connection tube 1 cannot be sealingly in contact any longer in the recess 15 of the first module 7 at least with the contact point 191 of the sealing bead 19.

The plug-type connection is thus no longer gastight as a whole. Thus, no reliable pneumatic connection is established in this special connection arrangement between the first and second modules 7, 8 through the connection tube 1 used, because the geometry of the connection tube 1 is not adapted to the position of and distance between the modules 6, 7, 8.

The first, second, third and fourth mounting possibilities or installation situations show that a secure and reliable pneumatic connection can be established between the first and second modules 7, 8 by means of the connection tubes 1 in case of slight deviations in the dimensional stability of the components or in case of deviations in the structural symmetry situation, especially in case of height offset 22 and/or an angular offset 23 between the first and second modules 7, 8 in relation to one another.

The fifth mounting possibility shows the structural limitations, where no reliable pneumatic connection can be established between the first and second modules 7, 8.

A secure and reliable pneumatic connection is made available by the connection tube 1 between two modules 6 of a respirator or anesthesia apparatus 4, 5. Only corresponding recesses 15 are to be formed during manufacture in the modules 6 of the respirator or anesthesia apparatus 4, 5. Only the connection tube 1 needs to be inserted into these recesses 15. The pneumatic connection can thus be established in a simple manner by plugging and it can also be detached in a simple manner, which is advantageous in case of maintenance work on the respirator or anesthesia apparatus 4, 5. Thus, only a very small number of components are advantageously needed for the pneumatic connection, so that the costs can be reduced, on the one hand, and the reliability of the mounting can be increased, on the other hand.

The connection tube 1 has, for example, a length 25 in the range of 25 mm to 35 mm and an external diameter 28 of about 8 mm to 12 mm, preferably an external diameter 28 of 10 mm.

Deviating from this, the length 25 of the connection tube 1 may be, for example, in the range of 10 mm to 40 mm and the diameter may be, for example, in the range of 5 mm to 15 mm. The length 25 depends essentially on the intended structural distance between the modules 6. The sealing bead thickness 29 depends on the elasticity and deformability of the elastic material of the sealing bead and is in a range of 1 mm to 5 mm. If a fluorinated rubber is selected as the elastic outer material and the elastic material of the sealing bead, a sealing bead thickness of about 2 mm is, for example, advantageous.

The height 26 and depth 24 of the recess 15 are selected such that a secure gastight, pneumatic connection is obtained in the interplay of the tube length 25, the sealing bead material and the sealing bead thickness 29 to the distance between the modules 6 within broad limits under the predetermined mounting situation.

The geometry or the size of the connection tube 1 may also be designed with respect to the recess 15 such that there is no risk of mix-up or incorrect plugging into unintended recesses 15 on a module 6 during mounting.

On the whole, substantial advantages are associated with the respirator or anesthesia apparatus 4, 5 according to the present invention. The pneumatic connection between the modules 6 of the respirator or anesthesia apparatus 4, 5 is substantially simplified, on the one hand, by the use of a smaller number of components, so that the manufacturing costs can be lowered and mounting errors can be essentially ruled out as a result. In addition, a highly reliable connection is made possible between the modules 6, so that the installation space needed for the respirator or anesthesia apparatus 4, 5 is advantageously reduced.

While specific embodiments of the invention have been described in detail to illustrate the application of the principles of the invention, it will be understood that the invention may be embodied otherwise without departing from such principles.

### APPENDIX

1 Connection tube
2 Outer tube consisting of outer material
3 Inner tube consisting of inner material
4 Respirator
5 Anesthesia apparatus
6 Module
7 First module
8 Second module
9 First end of connection tube
10 Second end of connection tube
11 Blower
12 Breathing system
13 Mixer
14 Anesthetic evaporator
15 Recess
16 Sealing surface
17 Air line in module
18 Flexible breathing tube
19 Sealing bead
20, 20′ Symmetry lines in the air lines 17
21 Symmetry line in connection tube 1
22 Height offset
23 Angular offset
24 Recess depth
25 Tube length
26 Recess height
27 Plug-in depth
28 Tube diameter
29 Sealing bead thickness
151, 152, 153, 154 Contact points of the recesses 15
191, 192, 193, 194 Contact points of the sealing beads 19

What is claimed is:

1. A respirator or anesthesia apparatus comprising:
at least two modules, at least one of the two modules being one of a blower module, a breathing system module, a mixer module and an anesthetic evaporator module;
at least one pneumatic connection means for a pneumatic connection of the at least two modules, the at least one pneumatic connection means comprising a connection tube surrounded at a first end by a first module of the at least two modules and surrounded at a second end by a second module of the at least two modules to provide the pneumatic connection between the first module and the second module by the connection tube.

2. A respirator or anesthesia apparatus in accordance with claim 1, wherein the connection tube is a separate component and/or not a flexible tube.

3. A respirator or anesthesia apparatus in accordance with claim 1, wherein the connection tube is connected to the first module and/or connected to the second module in a nonpositive or positive-locking manner.

4. A respirator or anesthesia apparatus in accordance with claim 1, wherein the first and/or the second module has a recess and the first end is arranged in the recess of the first module and/or the second end of the connection tube is arranged in the recess of the second module.

5. A respirator or anesthesia apparatus in accordance with claim 1, wherein the connection tube comprises a corrosion-resistant metal on an inside thereof and an elastic outer material on an outside thereof.

6. A respirator or anesthesia apparatus in accordance with claim 5, wherein the corrosion-resistant metal on the inside of the connection tube comprises stainless steel and the elastic outer material on the outside of the connection tube comprises an elastic material consisting essentially of one or more of a fluorinated rubber (FKM, FPM), a perfluorinated rubber (FFKM, FFPM) or an ethylene-propylene rubber (EPDM).

7. A respirator or anesthesia apparatus in accordance with claim 1, wherein the connection tube comprises a plastic on an inside thereof and an elastic outer material on an outside thereof.

8. A respirator or anesthesia apparatus in accordance with claim 7, wherein the plastic on the inside of the connection tube comprises a polyether ether ketone (PEEK) and the elastic outer material on the outside of the connection tube comprises an elastic material consisting essentially of a fluorinated rubber (FKM, FPM), a perfluorinated rubber (FFKM, FFPM) or an ethylene-propylene rubber (EPDM).

9. A respirator or anesthesia apparatus in accordance with claim 1, wherein:
the connection tube comprises an inner material and an outer material on an outside;
an elastic sealing bead is formed, with the outer material, at the first end and/or at the second end of the connection tube;
the sealing bead is provided in contact with the first and/or second module in a recess so that a sealing surface is formed between the sealing bead and the first and/or second module.

10. A respirator or anesthesia apparatus in accordance with claim 1, wherein the connection tube has a round cross section.

11. A respirator or anesthesia apparatus in accordance with claim 9, wherein a cross-sectional shape of the recess of the first module corresponds to an outer cross-sectional shape of the connection tube and/or to the sealing bead.

12. A respirator or anesthesia apparatus in accordance with claim 9, wherein the first end and/or the second end and/or the sealing bead of the connection tube is made slightly larger in a state in which the connection tube is not inserted into the recess of the modules than the recess in the modules so that the first end of the connection tube is arranged in the recess of the modules under a prestress.

13. A respirator or anesthesia apparatus comprising:
a first respirator or anesthesia module with a first module recess leading to a pneumatic connection opening;
a second respirator or anesthesia module with a second module recess leading to a pneumatic connection opening;
a pneumatic connection tube with a first end extending into the first module recess and surrounded by the first module and with a second end extending into the second module recess and surrounded by the second module to provide a pneumatic connection between the first module and the second module.

14. A respirator or anesthesia apparatus in accordance with claim 13, wherein:
the first module is one of a blower module, a breathing system module, a mixer module and an anesthetic evaporator module;
the second module is one of a blower module, a breathing system module, a mixer module and an anesthetic evaporator module; and
the connection tube is substantially inflexible and is a separate component from the first module and is a separate component from the second module.

16. A respirator or anesthesia apparatus in accordance with claim 14, wherein:
the connection tube comprises an integral structure with an outer tube portion formed of an outer tube material and an inner tube portion formed of an inner tube material;
the outer tube material is attached to the inner tube material by a connection in substance;
the inner material is a corrosion-resistant material; and
the outer material is an elastic material.

17. A respirator or anesthesia apparatus in accordance with claim 16, wherein:
a first elastic sealing bead is formed, with the outer material, at the first end;
a second elastic sealing bead is formed, with the outer material, at the second end of the connection tube;
the first sealing bead is provided in contact with an inner surface of the first module recess or a surface adjacent to the pneumatic connection opening of the first module; and
the second sealing bead is provided in contact with an inner surface of the second module recess or a surface adjacent to the pneumatic connection opening of the second module.

18. A respirator or anesthesia apparatus in accordance claim 17, wherein:
a cross-sectional shape of the first module recess corresponds to the outer cross-sectional shape of the connection tube and/or to the sealing bead; and
a cross-sectional shape of the second module recess corresponds to the outer cross-sectional shape of the connection tube and/or to the sealing bead.

19. A respirator or anesthesia apparatus in accordance with claim 17, wherein:
the first end or the first sealing bead is larger in a non connected state than an opening dimension of the first module recess so that upon the first end of the connection tube being arranged in the first module recess, the first end or the first sealing bead is compressed; and
the second end or the second sealing bead is larger in a non connected state than an opening dimension of the second module recess so that upon the second end of the connection tube being arranged in the first module recess, the second end or the second sealing bead is compressed.

20. A respirator or anesthesia apparatus comprising:
a first respirator or anesthesia module with a first module recess leading to a pneumatic connection opening, the first module being one of a blower module, a breathing system module, a mixer module and an anesthetic evaporator module;
a second respirator or anesthesia module with a second module recess leading to a pneumatic connection opening, the second module being one of a blower module, a breathing system module, a mixer module and an anesthetic evaporator module;
a pneumatic connection tube with a first end extending into the first module recess and surrounded by the first module and with a second end extending into the second module recess and surrounded by the second module to provide a pneumatic connection between the first module and the second module.
